(19) United States
(12) Patent Application Publication
(10) Pub. No.: US 2002/0102595 A1
(43) Pub. Date: Aug. 1, 2002

(54) Methods for detection of incorporation of a nucleotide onto a nucleic acid primer

(76) Inventor: Lloyd Mervyn Davis, Tullahoma, TN (US)

Correspondence Address: Richard T. Redano, Duane Morris LLP, One Greenway Plaza, Suite 500, Houston, TX, 77046, US

(21) Appl. No.: 10/059,754

(22) Filed: Jan. 29, 2002

Related U.S. Application Data
(60) Provisional application No. 60/264,790, filed on Jan. 29, 2001.

(51) Int. Cl. C12Q 1/68; C12M 1/34; G01J 3/42; G01J 3/427

(52) U.S. Cl. 435/6; 435/287.2; 356/319

(57) ABSTRACT

Methods and devices for detecting the incorporation of NTPs into immobilized enzyme-nucleic acid complexes are disclosed. The methods and devices can be used to genotype or sequence nucleic acids, including DNA and RNA, and are capable, in preferred embodiments, of detecting single incorporation events.

### FIELD OF THE INVENTION

The present invention is related to the detection of chemical reactions using optical detection methods. The present invention is particularly applicable to the detection of chemical reactions that involve nucleic acids.

### BACKGROUND OF THE INVENTION

Determination of the sequence of bases in DNA (“DNA sequencing”) can be accomplished by several techniques, including chain termination methods, and detection methods such as polyacrylamide gel electrophoresis and fluorescence measurements. DNA sequencing by synthesis is disclosed, for example, in U.S. Pat. Nos. 4,863,849 and 5,405,746. U.S. Pat. Nos. 5,302,509 and 6,255,083 disclose alternate methods for sequencing DNA or RNA during the replication or synthesis of an individual strand of polynucleic acid. In the alternate disclosed methods, a single polymerase enzyme is immobilized within a chamber containing solution; a single strand of DNA in the solution forms a complex with the immobilized enzyme; the complex is contacted with a solution containing labeled nucleotide triphosphates (NTPs); the enzyme then successively incorporates complementary nucleobases from the NTP solution into the single-stranded DNA (so as to form double stranded DNA); the DNA sequence is determined by the order in which nucleobases are incorporated from the solution onto the single_stranded DNA.

The detection of a single incorporation event (i.e., the incorporation of a single nucleobase) and of which nucleobase is incorporated at each step may be achieved, for example, by methods disclosed in U.S. Pat. No. 6,255,083. According to the disclosure of U.S. Pat. No. 6,255,083, after each incorporation event, a pyrophosphate (ppi) moiety is released into solution from the incorporated nucleobase. Each type of NTP in the solution is labeled by a different fluorescent label, the label being covalently bound to the ppi portion of the NTP. When the ppi is released from a nucleobase into solution, the fluorescent label, which is bound to the ppi, is also released and is detected optically. During optical detection, the color (or other distinguishable property) of the fluorescent label is noted in order to ascertain which of the four possible types of nucleobases was incorporated during that step. Thus in U.S. Pat. No. 6,255,083 nucleic acid sequencing is accomplished by detecting the incorporation of a labeled NTP into a single molecule of a target nucleic acid primer by detecting a unique label released from the labeled NTP. U.S. Pat. Nos. 6,232,075 and 6,306,607 disclose methods by which the fluorescently labeled ppi that results from an incorporation event may be distinguished from the fluorescently labeled NTPs that are free in solution, by covalently attaching fluorescence quenching molecules to the NTPs in solution. The fluorescence_quenching molecules are attached in close proximity to the fluorescent label and they become physically separated from the fluorescent label following an incorporation event, so that the signal from the fluorescent label increases. Thus an incorporation event is detected by a sudden increase in fluorescence signal from the vicinity of an immobilized enzyme_DNA complex. However, the incorporation of nucleobases onto a nucleic acid by an enzyme may be hindered by the simultaneous presence of a fluorescent label and a quenching molecule on the NTP.

A need remains for new techniques for detecting individual incorporation events while reducing the effects of detection and associated processes on the incorporation process, and on the DNA itself. The present invention provides methods and devices that allow the detection and identification of individual incorporation events, not by detecting the label released by the labeled NTP upon incorporation, but rather by detecting the labels before the incorporation has occurred. Furthermore, a quenching molecule is not required.

### SUMMARY OF THE INVENTION

The present invention provides methods and devices for optically detecting incorporation of a nucleotide into an immobilized complex comprising a single primer nucleic acid molecule and a single polymerase enzyme molecule. Such incorporation is referred to herein as an “incorporation event”. In preferred embodiments, the method provides optical detection of an incorporation event without the necessity of illuminating the complex and/or without the necessity of detecting the label while it is in contact with the complex.

According to one aspect of the invention, the method includes forming a stream of dilute solution comprising at least one type of nucleotide triphosphate (NTP), wherein at least one of said types of NTP is labeled with a detectable label, preferably a different detectable label for each type of NTP, and wherein each one of said labeled NTPs is serially ordered in said stream; passing said serially ordered labeled NTPs at a known speed through a first illumination zone upstream, preferably immediately upstream, of the immobilized complex, and then past the immobilized complex; passing said serially ordered labeled NTPs at a known speed through a second illumination zone downstream of the immobilized complex; detecting emitted light from said first illumination zone and determining from said emitted light the time at which each one of said labeled NTPs passes through said first illumination zone; determining, from the known speed of the labeled NTPs and from the time at which the labeled NTPs pass through the first illumination zone, a predicted time at which each labeled NTP would pass through the second illumination zone if the nucleotide did not become incorporated into the immobilized complex; detecting the presence or absence of emitted light from said second illumination zone at said predicted time; and determining from said presence or absence of light whether each labeled NTP detected passing through the first illumination zone is a labeled NTP of which the nucleobase has become incorporated into the nucleic acid complex.

In some embodiments, the label is a fluorescent molecule.

In some embodiments, the label is attached to the beta or gamma phosphate of the NTP.

In preferred embodiments, each labeled NTP is at a sufficiently dilute concentration that individual labeled NTPs may be detected and/or tracked.

In some embodiments, two or more distinguishable types of labels are used to label two or more different types of NTP. In preferred embodiments, detection of the labels in the first and/or second illumination zone includes distinguishing which type of label is detected, using color of excitation light or emission light, fluorescence lifetime, electrophoretic mobility, or one or more other distinguishable properties of the labels. Optical detection and detection of optical properties are preferred.

In some embodiments, an array of immobilized complexes is used and wherein optical detection of incorporation is separately accomplished for each complex of the array.

Another aspect of the invention is a method for genotyping a target nucleic acid. The method comprises forming a solution comprising at least one NTP, wherein said NTP is labeled with a detectable label; providing an immobilized complex comprising a target nucleic acid, a primer nucleic acid which complements a region of the target nucleic acid, and a nucleic acid polymerase enzyme molecule; causing said solution comprising said labeled NTP to pass at a known speed through a first illumination zone upstream of the immobilized complex; illuminating said labeled NTP with light at said first illumination zone, thereby causing said labeled NTP to emit light; detecting said light emitted by said NTP at the first illumination zone and determining from said light the time at which said labeled NTP passes through the first illumination zone; causing said solution comprising said labeled NTP to pass at a known speed past said immobilized complex, and through a second illumination zone downstream of said immobilized complex; determining, from the known speed of said labeled NTP and from the time at which said labeled NTP passed through the first illumination zone a predicted time at which said labeled NTP would pass through the second illumination zone if said nucleotide were not incorporated into said immobilized complex; detecting the amount of emitted light at said second illumination zone at said predicted time; and determining from said amount of emitted light at said predicted time that said nucleotide had become incorporated into the immobilized complex, to thereby genotype the target nucleic acid.

In some embodiments, the methods and devices disclosed here in can be used for sequencing a target nucleic acid.

Another aspect of the invention is a system for optically detecting incorporation of anucleotide into an immobilized complex comprising a primer nucleic acid molecule and a polymerase enzyme molecule, comprising:

(a) a solution flow chamber having a surface bearing the complex;

(b) a solution for contacting the complex, said solution containing one or more labeled NTPs;

(c) an illuminating device for illuminating at least two regions nearby the complex with light, said illuminating device providing a first illumination zone and a second illumination zone in said solution flow chamber;

(d) a transporting device for transporting the labeled NTPs through said first illumination zone, then past said complex, and then through said second illumination zone;

(e) an optical detection device for collecting light signals from the first illumination zone, and for determining the times at which each labeled molecule transits through the first illumination zone;

(f) an optical detection device for collecting light signals from the second illumination zone; and

(g) a computational device.

In preferred embodiments, the transporting device transports the labeled NTPs in a stream through the first illumination zone, and then through a zone containing the complex, and then through the second illumination zone, preferably at a known speed.

In some preferred embodiments, the optical detection device collects light signals from labels passing through each illumination zone. The optical detection device preferably includes a system for measuring the passage of labeled molecules through the first illumination zone. Also preferably, the optical detection device is capable of measuring the time at which labeled molecules pass through the first illumination zone.

In other preferred embodiments, the computational device includes a system for determining whether a label detected while passing through the first illumination zone was but is no longer attached to a NTP of which the nucleobase had become incorporated into the nucleic acid of an immobilized complex by computing a predicted time of passage of that label through the second illumination zone if it had not become incorporated into the nucleic acid, determining whether incorporation has occurred by determining whether an optical light signal is detected from the second illumination zone at the expected time.

In preferred embodiments, detection can be accomplished without illuminating the complex or detecting the label while it is in contact with the complex.

In some embodiments, an array of immobilized complexes is used, and the illumination zones extend upstream and downstream of the array. Preferably, the labeled NTPs within the stream of solution are individually imaged.

In some embodiments, the illumination zones move spatially over time. In other embodiments, the optical detection system for measuring the time of passage of labeled molecules through each illumination zone includes an imaging optical detection system or camera for resolving the location of each labeled molecule at the point of its detection.

In some embodiments, the means for spatially moving the illumination zones comprises one or more moveable slits and a stationary wire, which can be positioned to occlude a substantial area of the focused laser beam at the first and/or second illumination zone. In other embodiments, the means for spatially moving the illumination zones comprises an acousto-optic beam-steering device, which deflects one or more laser beams to a point in the illumination zone that moves spatially with time.

In some embodiments, two or more colors of light are used for illumination at different locations. The location of a labeled molecule at the point of detection may be used to identify the label.

These and other aspects of the invention will be apparent to one skilled in the art in view of the following disclosure and the appended claims.

### BRIEF DESCRIPTION OF THE DRAWING

FIG. 1 is a system for the optical detection of incorporation events, including a laser beam source, optical detector, lenses and other optical components.

FIG. 2 is a photographic and graphic depiction of fluorescence emission from molecules, according to an embodiment of the invention.

FIG. 3 is a side view of a system for imaging of labeled molecules and determination of the time of passage of the molecules at a selected location.

FIG. 4 is a one-dimensional graphical depiction of a “time line” of a labeled NTP wherein position of the NTP in one dimension is plotted versus time.

### DETAILED DESCRIPTION

The present invention provides methods and devices for optical detection and identification of chemical reactions. The methods and devices described herein are particularly applicable to the detection of chemical reactions in which the nucleobase from a labeled NTP becomes incorporated onto a nucleic acid by the action of a polymerase enzyme. Such reactions are referred to as “incorporation events”. However, the methods and devices described herein can be applied to the detection of other chemical reactions, when such detection can be facilitated by the optical properties of single molecules.

While it is not intended that the present invention be bound by any particular theory or mechanism, it is believed that the methods and devices disclosed herein are aided by the retention of the fluorescent label of a labeled NTP at the site of incorporation of the NTP for a brief time during an incorporation event. There are a series of steps that are thought to occur when an enzyme incorporates a nucleobase onto a complementary position of a DNA strand in an immobilized enzyme-DNA complex. In a simplified view, the steps can be reduced to the following. First, a complementary NTP is transported by diffusion into the immediate vicinity of the immobilized enzyme-DNA complex (step 1). When the NTP is in position, the polymerase molecular conformation changes. If the polymerase is assumed to be shaped like a minuscule hand, in the conformation change, the thumb closes over the NTP as the nucleobase makes chemical bonds with the DNA and breaks bonds with the pyrophosphate (ppi) of the NTP (step 2). The ppi (and fluorescent label) is then free to be transported away into solution by diffusion and/or other processes, while the enzyme “thumb” opens and the enzyme moves to the next DNA site (step 3).

During the configuration change of the polymerase, a single complementary NTP together with ppi and fluorescent label is momentarily held stationary at the site of the immobilized enzyme-DNA complex. The label is not free to be transported away from the site by diffusion and/or other processes, as the NTP is physically constrained by the enzyme. The amount of time for which the fluorescently labeled ppi is constrained by the enzyme so that it cannot be transported away can be estimated from bulk studies of the rate for enzyme incorporation, which follows Michaelis-Mentin enzyme kinetics. At sufficiently high concentrations of free NTPs, the rate of incorporation asymptotically approaches a maximum value, Vₘₐₓ, which is usually about 300 incorporations per second or less. Under these conditions, step (1) occurs very quickly compared to steps (2) and (3). Therefore, the time required for steps (2) and (3) is about 1/300 of a second, or about 3 milliseconds. The extent of the conformational changes in the enzyme that occur during steps (2) and (3) are comparable, and thus it may be estimated that the minimum time required for step (2) is about 1.5 milliseconds. This minimum time for which the fluorescently labeled NTP is momentarily held stationary could be greater than 1.5 milliseconds if other solution conditions were appropriately selected.

Unless otherwise stated, the terms below defined, when used herein, have the definitions set forth herein.

“Nucleobase” means one of the purine or pyrimidine derivatives that are components of nucleotides of nucleic acids, i.e. adenine, thymine, guanine, cystein, uracil.

“Nucleotide” means a structural unit of a nucleic acid, which is an ester of a nucleoside and phosphoric acid.

“Nucleoside” means a glycoside, comprising a pentose sugar linked to a purine or pyrimidine nucleobase.

“Primer” means a short double-stranded nucleic acid sequence that has a 3′—OH terminus at which a DNA polymerase can begin synthesis of a nucleic acid chain.

“Serially ordered”, when used to refer to NTPs, means that the NTPs are disposed in a particular order with respect to each other, which they retain as they flow in solution according to the methods described herein.

“Immediately”, when used to denote the location of an illumination zone with respect to an immobilized complex, means that sufficiently far from the enzyme-nucleic acid complex that light irradiation of the complex is minimized. Preferably, the distance between the illumination zone and the immobilized complex is large enough that the complex is not irradiated by light from the illumination zone.

“Incorporation” and “incorporation event”, when used herein to refer to interaction between an immobilized complex and an NTP, mean that the phosphate from the NTP is cleaved, leaving a nucleobase, which becomes incorporated into the primer nucleic acid of the immobilized complex.

To “sequence” means to determine the type and order of successive nucleobases along a strand of a nucleic acid.

To “genotype” means to determine that some of the sequence of a part of a nucleic acid is essentially the same as that of a known gene.

“Nearby”, when used to indicate the relative location of illuminated regions and an immobilized nucleic acid complex means within a distance of about 5 microns, preferably about 1 micron.

The fact that the detailed structure of nucleic acids, such as DNA, is much smaller than an optical wavelength generally precludes the detection of the fine detailed structure of an enzyme-nucleic acid complex in order to optically resolve a NTP that is undergoing incorporation from one that merely passes by the vicinity of the complex without undergoing incorporation, even with the best currently available optical resolution. The present invention, however, provides methods for distinguishing a NTP that participates in incorporation from one that passes through the smallest optically resolvable volume at the enzyme-nucleic acid complex without becoming incorporated.

The present invention provides methods for determining whether a nucleotide becomes incorporated into a nucleic acid. A preferred method includes: forming a solution comprising at least one NTP, wherein said NTP is labeled with a detectable label; providing an immobilized complex comprising a primer nucleic acid molecule and a polymerase enzyme molecule; causing said solution comprising said labeled NTP to pass at a known speed through a first illumination zone upstream of the immobilized complex; illuminating said labeled NTP with light at said first illumination zone, thereby causing said labeled NTP to emit light; detecting said light emitted by said NTP at the first illumination zone and determining from said light the time at which said labeled NTP passes through the first illumination zone; causing said solution comprising said labeled NTP to pass at a known speed past said immobilized complex, and through a second illumination zone downstream of said immobilized complex; determining, from the known speed of said labeled NTP and from the time at which said labeled NTP passed through the first illumination zone a predicted time at which said labeled NTP would pass through the second illumination zone if said nucleotide were not incorporated into said immobilized complex; detecting the presence or an absence of emitted light from said second illumination zone at said predicted time; and determining from said presence or absence of emitted light at said predicted time whether said nucleotide had become incorporated into the immobilized complex. The present invention also provides methods for identifying which of the possible types of NTP is incorporated into a nucleic acid, by using different labels for each type of NTP and by determining the identity of each labeled NTP as it passes through the first illumination zone.

The methods and devices disclosed herein also can be used to sequence a nucleic acid. In sequencing a nucleic acid, the identity as well as the order of successive nucleobases along a strand in the nucleic acid are determined. According to the methods and devices disclosed herein, sequencing a nucleic acid further comprises sequentially repeating the steps for detecting the incorporation of a single NTP, wherein the detecting step also includes identifying which of the 4 possible types of NTP is incorporated.

The present invention further provides methods for genotyping target nucleic acids. A preferred method for genotyping a target nucleic acid comprises determining the sequence of a part of a nucleic acid and comparing the sequence with that of a known gene.

The present invention also provides methods and devices for the detection of individual incorporation events in which a label of a labeled NTP participating in an incorporation event with an enzyme-nucleic acid complex is detected while not in contact with the enzyme-nucleic acid complex, e.g., when the label has been released from the enzyme-nucleic acid complex, and in which the enzyme-nucleic acid complex preferably undergoes no optical irradiation. Preferred labels for use in the methods and devices described herein are fluorescent labels. Avoidance of irradiation of the enzyme-nucleic acid complex and detection of the label while not in contact with the enzyme is preferred for several reasons. First, the absorption of light by an enzyme can cause irreversible photodamage to the enzyme, as described, for example, in Yin, et al., Science 270, 1653-1657 (1995), and Ref. 20 therein. In the Yin study, immobilized enzyme_nucleic acid complexes were found to suffer irreversible photodamage after 82±58 seconds of irradiation by a 82-99 milliwatt neodymium/yttrium/lithium fluoride 1047 nm laser beam that was focused on a bead near the enzyme. Photodamage could occur more quickly if the enzyme were directly irradiated by a laser beam, particularly if visible wavelengths were used, as would be required for linear excitation of visible fluorescent labels. Second, the enzyme is generally larger than the fluorescent label and can give rise to Rayleigh and Raman light scattering that may make it more difficult to detect a single fluorescent label in the immediate vicinity of the enzyme. Lower background and higher efficiency of detection of single fluorescent labels is likely at locations away from the enzyme. Third, the enzyme may quench the fluorescence of a label when the label is embedded within the enzyme during an incorporation event, for example, due to the formation of a charge-transfer complex. Fourth, if the fluorescent label were to become photobleached in the immediate vicinity of the enzyme, the label could form chemically reactive radicals that could damage the enzyme.

According to the methods of the present invention, an enzyme-nucleic acid complex is immobilized onto a solid surface. The surface containing the enzyme is contacted by a flowing solution containing one or more types of fluorescently labeled NTPs. “Types of NTPs” means NTPs bearing different nucleobases. The different nucleobases may be selected from those known to one skilled in the art, including cytosine, uracil, guanine, and thymine, xanthine, hypoxanthine, methylated cytosine, and derivatives thereof. Two or more different NTPs may be used. The NTPs may be labeled with a detectable label, such as, for example, a fluorophore. A “detectable label” is a chemical substituent or group that is capable of emitting a signal, such as light, that can be measured by conventional laboratory detection equipment, such as diode arrays, cameras, and the like. Fluorescent labels, i.e., molecules, moieties, or substituents that emit flourescent light when irradiated, are preferred. However, labels that permit the use of other light-induced phenomena, such as Raman scattering and semiconductor luminescence may be used. Two or more fluorophores of different colors, or having other measurably different properties, such as, for example, fluorescence lifetime or electrophoretic mobility, may be used, so that the type of NTP that participates in an incorporation event may also be identified.

While it is not intended that the present invention be bound by any particular theory or mechanism, it is believed that, in accordance with the methods disclosed herein, the transport of NTPs in the vicinity of an immobilized enzyme-DNA complex is dominated by mechanism(s) other than diffusion, such as, for example, electrokinetic or hydrostatic forces, which can produce a steady bulk flow. Laminar flow is highly preferred, so that that the NTPs remain serially ordered as they flow in the solution past the immobilized complex. Flow facilitated by other means, such as gravity or pressure, is also useful, provided that the motion of the NTPs is not dominated by diffusion and NTPs which are initially serially ordered remain serially ordered following an incorporation event at least until the label of the labeled NTP passes a first illumination zone and a second illumination zone.

The methods of the present invention utilize solutions of labeled NTPs that are preferably sufficiently dilute that each NTP may be individually tracked or detected optically. By “tracked” is meant followed over time. For example, it is preferred that the concentration be 10⁻⁸ M (moles per liter, or “molar”) or less, more preferably 10⁻⁹ M or less, and even more preferably 10⁻¹⁰ M or less. Although no particular minimum concentration is required, the practical lower concentration may be effected by fluorescent impurity concentration limits, as the methods described herein involve detecting single molecules. The solution can be made using aqueous buffers containing salts, such as Mg2+salts, in concentrations adequate to control enzymatic reactions. Appropriate composition and concentration of buffer solutions for use in making NTP solutions may be determined by one skilled in the art.

The methods of the invention also utilize an immobilized nucleic acid-enzyme complex comprising a primer nucleic acid and an enzyme. Preferably the complex comprises a single nucleic acid molecule and a single enzyme molecule. Preferred enzymes are polymerases, such as DNA polymerase or RNA polymerase. The complex is contained within a sample chamber, the sample chamber having a void through which a solution can flow and a surface surrounding the void, and immobilized onto the surface of the sample chamber. The NTP solution is transported through the void by, for example, an electro-osmotic flow, or by a peristaltic or other type of pump or gravitational flow. It is preferred that the solution be transported by electro-osmotic forces, which result in a “plug flow”, such that the flow velocity is substantially the same at all points in any cross section of the void. The flow rate is not critical; however, a flow rate of about 5 microns (micrometers, 10⁻⁶ meter) per millisecond (10⁻³ second) has been found to be suitable. Faster flow rates are preferable, as faster flow rates minimize uncertainties in the expected detection times of signals at the second illumination zone due to diffusion. However, faster flow rates will result in a faster transit time of labels through the illumination zones and hence a lower signal from labels. Single molecule detection of fluorescent labels has been achieved with transit times less than 100 microseconds (see later). For an illumination zone of 0.5 microns diameter, this corresponds to a flow velocity of 5 microns per millisecond (5 millimeters per second). With improvements in the instrument, flow velocities of 50 microns per millisecond should be possible. It is preferred that the flow rate be substantially steady, i.e. not fluctuate more than about 10percent.

The solution is carried into the chamber through a first illumination zone, which is located upstream from the immobilized enzyme-nucleic acid complex. Preferably, the first illumination zone is immediately upstream from the immobilized enzyme-nucleic acid complex, preferably about 0.5 to about 5 microns from the enzyme-nucleic acid complex. Preferably, the complex receives substantially no irradiation. Light emitted by the NTPs is collected from the first illumination zone and the time at which each fluorescence light signal is collected, which corresponds to the time at which each labeled NTP passes through the first illumination zone, is recorded.

The size of the illumination zone is not critical, and will be determined in part by the size of the light source used for illumination, e.g., the diameter of a laser beam when focussed into the solution. The size and location of the first illumination zone is preferably such that all labels that pass within less than about 1 micron of the complex will have passed through the first illumination zone and will have been detected. Similarly, the size and location of the second illumination zone is such that all labels that have passed through the volume nearby to the complex will pass through the second illumination zone and will be detected. Different types of fluorescent labels may be used to label each type of NTP and thus the color or other property of the light detected from each label may be used to determine the type of each detected NTP.

The source of light used to illuminate the illumination zones is preferably a laser, as the coherence properties of the laser enable the light to be focused to a selected region within a fraction of a wavelength, i.e. less than about one micron from a selected region. The laser source may provide either pulsed or continuous light. The wavelength of laser light is not critical, and can be selected by one skilled in the art in view of the optical properties of the molecule(s) to be illuminated.

The NTP solution passes from the first illumination zone to a volume immediately surrounding the immobilized complex. The NTPs that do not become incorporated then pass to a second illumination zone, immediately downstream from the immobilized complex. Preferably, the second illumination zone is sufficiently far from the complex that the complex undergoes substantially no irradiation.

The methods and devices of the present invention are useful for detecting incorporation, for example, when the labels are attached to the ppi moiety of an NTP, or when the labels are attached directly to the nucleobases. If the labels are attached to the nucleobases, and if any one nucleobase becomes incorporated into the DNA by the enzyme, then the label for that nucleobase will not be detected at the second illumination zone, as it will remain attached to the incorporated nucleobase. On the other hand, if the labels are attached to the ppi moieties of the NTPs, for example, if incorporation of a nucleobase into the complex occurs, the label and ppi for that nucleobase will be momentarily held stationary. Complexes in which labels are attached to the beta or gamma-phosphate of a NTP are disclosed in U.S. Pat. Nos. 6,232,075 and 6,306,607, the disclosures of which are hereby incorporated herein in their entirety. In such complexes, the label transits the second illumination zone but fluorescence is not detected at the time that is expected in the absence of incorporation.

According to the methods of the invention, incorporation of a single nucleobase onto an immobilized complex comprising a primer nucleic acid and an enzyme is detected by: (a) contacting the complex with a solution containing one or more labeled NTPs comprising a label that emits light upon irradiation, said solution flowing in contact with the complex at a known flow speed; (b) determining the passage of each labeled NTP at a selected location upstream of the complex by collecting and measuring emission of light from the labeled NTP; (c) determining and recording the time at which each such passage occurs; (d) calculating, for each labeled NTP, from each such measured passage time and the known flow speed, the expected time at which each labeled NTP would pass a selected downstream location of the complex if the NTP did not become incorporated into the DNA of the complex; and (e) determining whether the nucleobase of each labeled NTP detected passing through the first upstream illumination zone did or did not become incorporated into the DNA of the immobilized complex, based on the absence or presence of emitted light from the labeled NTP at the calculated expected time at the selected downstream location.

Also within the scope of the present invention are systems for optically detecting incorporation of nucleotide into immobilized complexes comprising a primer nucleic acid molecule and a polymerase enzyme molecule. A preferred embodiment comprises:

(a) a solution flow chamber having a surface bearing the complex;

(b) a solution for contacting the complex, said solution containing one or more labeled NTPs;

(c) an illuminating device for illuminating at least two regions nearby the complex with light, said illuminating device providing a first illumination zone and a second illumination zone in said solution flow chamber;

(d) a transporting device for transporting the labeled NTPs through said first illumination zone, then past said complex, and then through said second illumination zone;

(e) an optical detection device for collecting light signals from the first illumination zone, and for determining the times at which each labeled molecule transits through the first illumination zone;

(f) an optical detection device for collecting light signals from the second illumination zone; and

(g) a computational device.

The computational device is capable of, and uses appropriate software for, determining when light signals are expected from the second illumination zone from labeled molecules that were detected while transiting the first illumination zone, and determining whether in fact such light signals occur at all, and/or whether the light signals occur when they are expected. The presence of a light signal of a particular wavelength corresponding to the wavelength of light that is expected to be emitted by a label on a labeled NTP at the expected time indicates that incorporation of the labeled NTP has not occurred. The absence of a light signal of a particular wavelength corresponding to the wavelength of light that is expected to be emitted by a label on a labeled NTP at the expected time indicates that incorporation of the labeled NTP has occurred. If desired, the computational device can be programmed and equipped to provide, in conjunction with optical detection devices, graphical and/or tabular output indicating the wavelength of light emitted at the second illumination zone and/or the time at which light of a particular wavelength was emitted.

“Upstream”, as used herein to refer to the flow of solutions containing NTPs, means at a location past which the solution would flow before flowing past the immobilized DNA complex.

“Downstream”, as used herein to refer to the flow of solutions containing NTPs, means at a location past that which the solution would flow after flowing past the immobilized DNA complex. The present invention provides methods and devices that include detecting individual labeled molecules in solution. Suitable techniques for detection of fluorescence from molecules are known to those skilled in the art. Preferred embodiments are described in accordance with the Figures. The preferred embodiments are intended as illustrative, but do not limit the scope of the present invention.

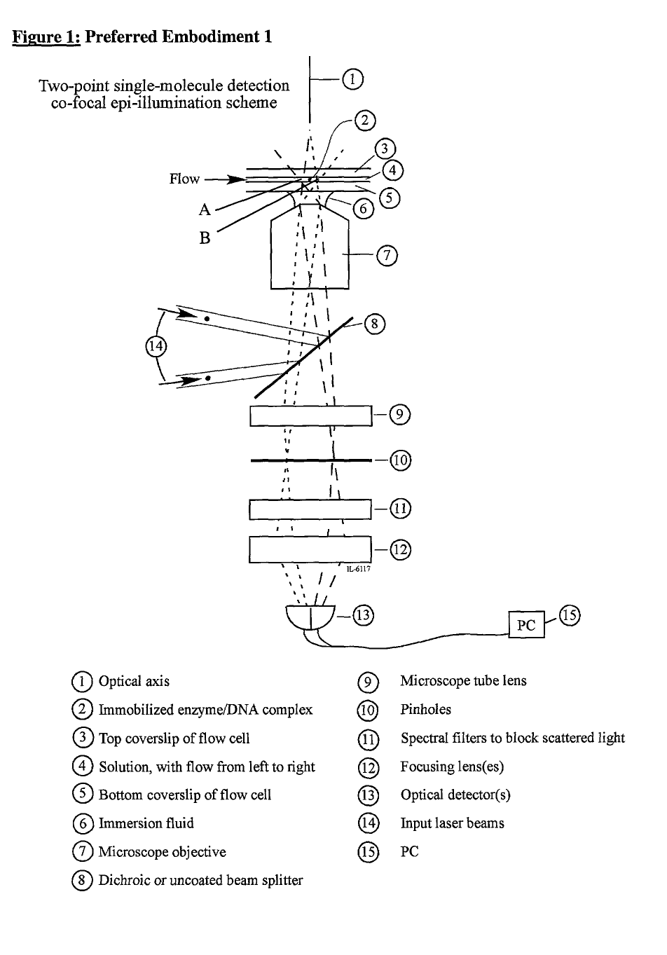

A preferred embodiment of the present invention is described with reference to FIG. 1. Two laser beams (14) enter at a dichroic beam splitter (8) at angles slightly inclined to the optical axis (1) of a microscope objective (7), coupled via immersion fluid (6) to a flow chamber, comprising a transparent bottom coverslip (5), a solution chamber (4), and a top coverslip (3), which may or may not be transparent. A solution containing labeled NTPs flows in the flow chamber, from left to right as shown in FIG. 1, at a known speed, v. The two laser beams are focused to two spots or illumination zones (A) and (B), which are located immediately upstream and downstream of the immobilized enzyme/DNA complex (2), so that the immobilized complex suffers no laser illumination. The centers of the two illumination zones are separated by a distance d. Emitted light (fluorescence) from labeled molecules passing through each illumination zone is collected by the microscope objective (7), and is imaged by the microscope tube lens (9) to two disks conjugate to the illumination zones and coincident with two pinholes in the image plane (10) of the microscope. The pinholes serve to spatially filter the collection of light so that each pinhole only passes light that emanates from the respective illumination zone. From the pinholes, the collected fluorescence light passes through spectral filters (11) to block scattered light, and is focused using one or more focusing lenses (12) onto one or more optical detectors (13). The signals from the detectors pass to electronics and a computer (15) for analysis.

When a labeled NTP passes through the upstream illumination zone, one of the optical detectors will receive a signal consisting essentially of one or more photons of fluorescence light. The centroid of the times of detection of the photons, t1, is evaluated by the computer, and the expected time of passage through the downstream illumination zone for a label that has not participated in an incorporation event is calculated as t2=t1+v d. The computer can be programmed to provide selected output to indicate whether the labeled NTP detected in passing through the upstream illumination zone had become incorporated into the DNA, based on whether an optical signal was detected from the downstream illumination zone by the corresponding optical detector centered at time t2. If there was no optical signal detected that was centered a time t2, then the computer indicates that incorporation of a nucleobase onto the DNA has occurred.

Parameters that can be varied by one skilled in the art in carrying out the methods described herein include the resolution of the optics before the optical detector, irradiance of the light, and tightness of focus of the laser beam. Preferably, the irradiance is such that the labels of the labeled molecules are excited, upon irradiation, to an extent less than saturation. For example, a power on the order of microwatts (1 microwatt=10⁻⁶ watt), such as, for example, about 10 to 100 microwatts, is generally preferred. However, as the laser beam is focused less tightly, a higher power, such as about 10 to about 50 milliwatts, may be preferred.

The accuracy of detection of incorporation events can be improved by selection and/or control of flow rate and illumination conditions. Preferred conditions are determined, in part, by molecular diffusion.

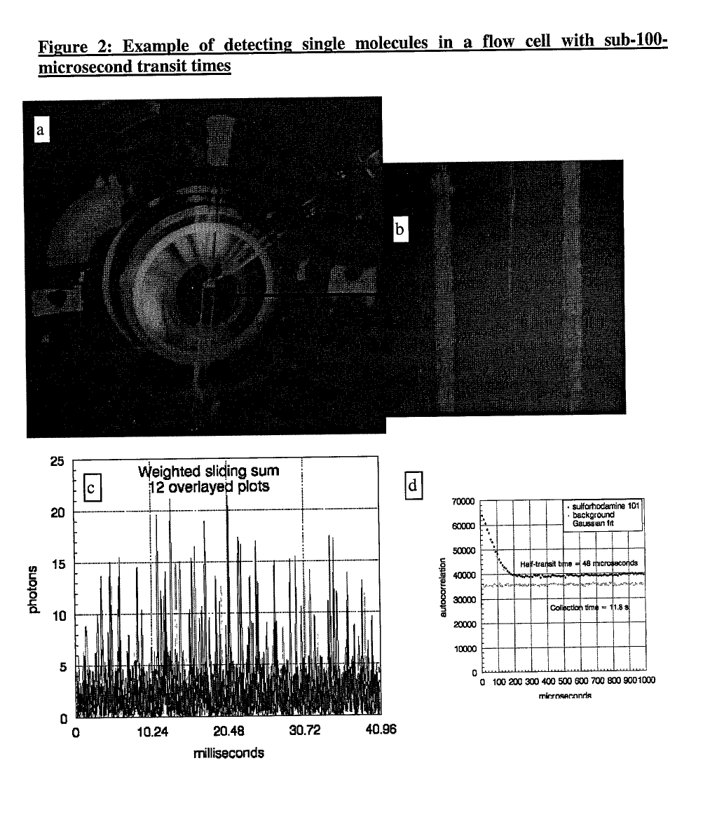

In preferred embodiments, the methods of the present invention overcome the effects of diffusion on the detectability of incorporation events by minimizing the effect of diffusion on the transport of labeled NTPs. The following example is provided for illustrative purposes regarding the effects of diffusion. The diffusion coefficient for labeled NTPs and labeled ppi moieties can be represented as D=2.5×10⁻¹⁰ m²/s. This is a worst_case calculation, because the diffusion coefficient may be smaller in flow chambers with sub_micron dimensions than in flow chambers with dimensions larger than several microns. Thus, sub-micron chambers are preferred. The diffusion coefficient is also smaller in solutions at temperatures below room temperature or containing a viscous component such as glycerol. The focal spots of the laser beams at the first illumination zone and the second illumination zone (A and B in FIG. 1) are each elliptical, oriented with their semi_minor diameter in the direction of the flow. The elliptical laser spots have semi-minor diameters of 0.5 microns, and centers separated by d=1.5 microns. For a labeled NTP that passes the immobilized enzyme-nucleic acid complex without participating in an incorporation event, the mean time Δt between the centroids of the optical signals from the two illumination zones is Δt=d/v, and the root-mean-square (rms) distance of diffusion during the mean time in any dimension is √(2DΔt). Therefore the rms variation in the time between the two centroids is σ_{Δt}=√(2DΔt)/v=√(2Dd/v³). For given values of σ_{Δt}, d, and D, the flow velocity is v=[(2Dd)/σ_{Δt}²]^{⅓}. This equation sets the minimum flow velocity that may be used if the uncertainty in the expected detection time of a labeled NTP in the second downstream illumination region is to be smaller than the residence time of the label at the incorporation site. For example, in order to be able to discern a 1 standard deviation difference in delay of σ_{Δt}=0.1 ms, the flow speed is v=[(2×2.5×10⁻¹⁰ m²/s×1.5×10⁻⁶ m/10⁻⁸ s²]=4.2×10⁻³ m/s, or faster. The transit time of the label across either illumination zone is 120 microseconds, which can be measured using the devices and methods described herein. For example, transit times of at least about 100 microseconds are measurable using the methods described herein. Detection of single molecules within sub−100 microsecond transit times has been experimentally demonstrated even using relatively low collection efficiency optics with numerical aperture N.A.=0.85, as shown for example in FIG. 2. FIG. 2a is a photograph of a flow cell in front of a microscope objective; FIG. 2b is an image obtained with an intensified camera and long exposure time showing a bright fluorescent spot caused by many molecules transiting through the focused laser beam; FIG. 2c shows a graph of the number of detected fluorescence photons versus time, in which individual peaks of 5—25 photons result from the transit of single molecules of sulforhodamine 101 through the focused laser beam; and FIG. 2d shows the autocorrelation function of the photon stream, from which it is evident that the transit time of single molecules is less than 100 microseconds. Slower flow rates can be used if the diffusion coefficient is smaller and/or the required precision σ_{Δt} is larger. For example, the presence in the solution of components such as glycerol, which can inhibit diffusion, can provide smaller diffusion coefficients and allow the use of lower flow rates. Also, smaller diffusion coefficients can provide greater precision. Determination of appropriate flow rates for the detection characteristics of the components used and the composition of the solution may be accomplished by one skilled in the art.

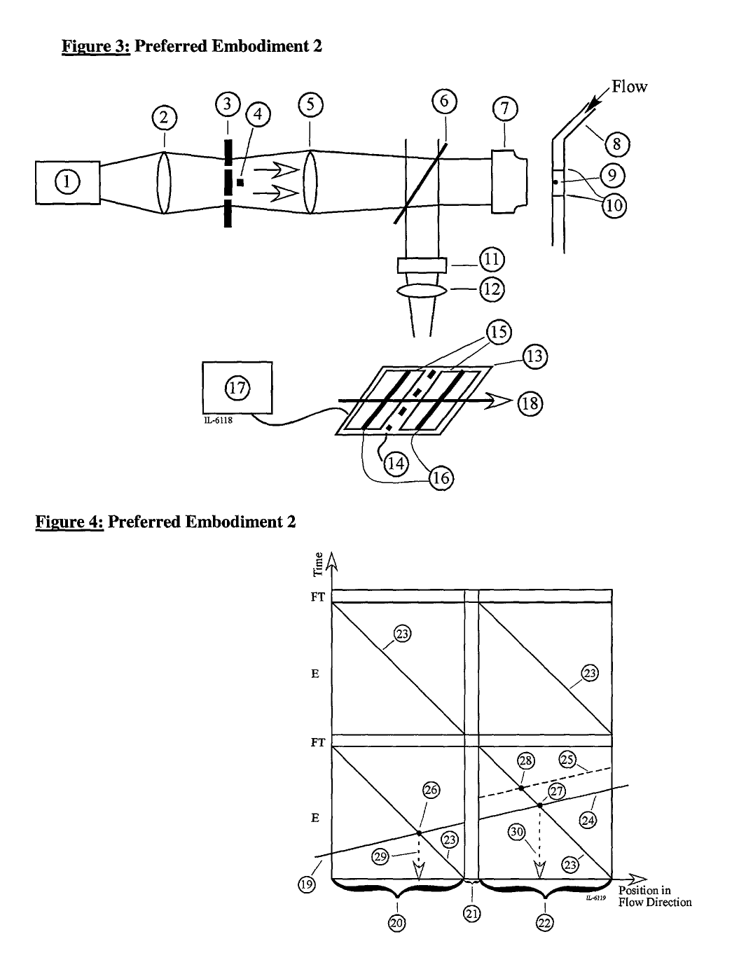

A second preferred embodiment is illustrated with reference to FIG. 3. A flow chamber (8) contains a linear array (9) of immobilized enzyme-DNA complexes. The flow chamber (8) is imaged onto the focal plane of a frame-shift camera (13), such as Roper Scientific's Micromax EEV back-illuminated 512×512 pixel camera, with minimum exposure time of 350 ms and frame shift time of <10 ms. The system in FIG. 3 permits imaging of labeled molecules and determination of the time of passage of the molecules at a selected location to a precision much shorter than the camera exposure time.

As shown in FIG. 3, the beam from a laser (1) is imaged by a lens (2) to a plane at which one or more moving slits (3) and a stationary wire (4) are positioned so as to occlude all but one or more small slices of the beam. As the slits move, the slices of the laser beam that are not occluded also move. The non_occluded portions of the beam then pass through a tube lens (5), dichroic filter (6), and microscope objective (7). The tube lens and microscope objective image the non_occluded portions of the beam onto one or two lines of illumination (10) within the flow cell (8) in zones on either side of the linear array of immobilized enzyme_DNA complexes (9), which is oriented transverse to the flow and into the plane of the page in FIG. 3. The stationary wire (4) is imaged along the array (9) and blocks the beam from the array so that the array is not illuminated, thereby avoiding possible radiation damage to the enzymes in the array (9) of complexes. The moving slits (3) are imaged into the flow chamber (8) to provide moving lines of laser illumination (10) that scan the flow chamber (8) above and below the immobilized array in a direction counter to the flow of labeled NTPs in solution. In FIG. 3, the lines of laser illumination (10) are oriented into the page.

FIG. 3 illustrates one exemplary method for providing a line of illumination that can scan across regions extending above and/or below an immobilized array. By one skilled in the art of optics, other optical configurations can be set up to achieve the same end. In particular, optical detectors containing acousto-optic components, such as, for example, components used in laser light shows, can also be configured to cause thin lines of illumination to be scanned across an extended region. A system based on acousto-optics could be advantageous in some applications because it can provide increased efficiency in the use of the laser power, in contrast with techniques using moving slits and wires, which result in a part of the laser beam being occluded.

In the embodiment shown in FIG. 3, fluorescence light is collected from labeled molecules in the flow chamber (8) by the objective (7), reflected by the dichroic filter (6), passed through a spectral filter (11), and imaged by a tube lens (12) onto the focal plane of the camera (13). No image of the immobilized array (9), appears at the camera focal plane at location (14), because the immobilized array is not illuminated. However, on either side of location (14), images of the upstream and downstream illumination zones appear as rectangular regions (15). At a particular time, only thin lines of the illuminated region of the immobilized complex undergo laser excitation and thereby emit fluorescence light onto the camera focal plane. In FIG. 3 the lines of illumination are imaged to lines at locations (16).

The camera images are transferred to a computer (17) for analysis. In the exemplified embodiment, a labeled NTP molecule that is carried by the solution flows along a straight_line trajectory, which is imaged to the path (18) in FIG. 2. Only the motion of the labeled NTP in the direction of the flow is considered in the exemplified embodiment. The “time line” of the labeled NTP may be graphically represented as in FIG. 4, wherein position in one dimension, i.e., the direction of flow, is plotted versus time.

The labeled NTP “time line” appears as a diagonal line (19) with positive slope. The images of the upstream illumination zone, the non-illuminated zone around the immobilized enzyme_DNA complex, and the downstream illumination zone are labeled (20), (21), and (22) respectively. Along the vertical time axis, the times at which the camera exposure shutter is open (E) and at which the shutter is closed during frame transfer (FT) are shown. The line of laser illumination is scanned across the downstream and upstream illumination zones at a constant speed in a direction counter to the flow. The “time line” of the image of the line of laser illumination (in one dimension along the line (18)) appears as diagonal lines (23) in FIG. 4.

Each zone is fully scanned in a time that is equal to or shorter than the camera exposure time. Therefore, the “time line” of a labeled molecule intersects the “time line” of the laser illumination line at at least one point in each illumination zone.

Labeled NTPs enter the upstream illumination zone at times that are random with respect to the scan of the laser line. Thus the “time line” of a labeled NTP may be any line parallel to line (19) of FIG. 3. If the labeled NTP does not participate in an incorporation event, but continues to be carried at a constant speed into the downstream illumination zone, then its “time line” continues undeviated in a straight line (24). However, if the labeled NTP does participate in an incorporation event, the label is either momentarily or permanently held stationary, depending (respectively) on whether the label is attached to the nucleobase or the ppi moiety. If the label is attached to the nucleobase, the label is not detected in the downstream illumination zone. If the label is attached to the ppi moiety, the “time line” of the label through the downstream illumination zone is shifted as shown at (25).

The points of intersection of the “time line” of a labeled molecule and the “time line” of the laser line, (26), and (27) or (28), indicate the precise location of the labeled molecule within each illumination zone for a given camera frame image. That is, the scanning laser illumination line effectively takes a snapshot of the location of the labeled molecule at a particular time. If a labeled NTP image is obtained in the upstream illumination zone at the location (29) corresponding to the intersection point (26), and if the NTP did not participate in an incorporation event, then in the next camera frame image the image of a label appears in the downstream illumination zone at the precise location (30) corresponding to the intersection point (27). The absence of an image at location (30) indicates that the nucleobase of the labeled NTP has become incorporated into the DNA of the immobilized complex.

For example, if the optical resolution is such that a point source, such as a single molecule, creates an image that appears to be 0.5 microns in diameter (i.e., the Airy disk in object space is 0.5 microns), and if the magnification is such that the Airy disk is mapped to a single pixel on the 512×512 camera, and each illumination zone extends 125 microns in width (along the direction of flow) and that the dark zone around the immobilized array is 6 microns in width, then as seen by the camera each illumination zone is 250 pixels wide, the dark zone is 12 pixels wide, and the total image is 512 pixels wide. For a labeled NTP that does not incorporate, the points of detection in each illumination zone are to be separated by 250+12=262 pixels or 131 microns. Suppose the diffusion coefficient is D=2.5×10⁻¹⁰ m²/s. (This is a worst_case” calculation, because the diffusion coefficient will be smaller in flow chambers with sub_micron dimensions or in solutions at temperatures below room temperature or containing a viscous component such as glycerol.) Suppose the flow speed is v=10⁻² m/s. Then the time taken for molecules to travel 131 microns is Δt=0.0131 s, and the time taken to travel 1 pixel or 0.5 microns is 50 microseconds. The rms fluctuation due to diffusion in the time taken to travel 131 microns is √(2DΔt)/v=2.56×10⁻⁴ s. If, for example, the laser scan speed is set at 1 pixel/2.56×10⁻⁴ s or 250 pixels/0.064 s, which corresponds to 0.5 Υ [symbol] \ddot{Υ}′ωφ,/2.56×10 ⁻⁴ s=1.95×10⁻³ m/s, then the rms fluctuation due to diffusion in the separation of the points of detection of a molecule would be ±1 pixel. If a label had remained stationary at the immobilized complex for 1.5 ms, the laser would have scanned a distance of 1.5×10⁻³ s×1.95×10⁻³ m/s=2.925 microns corresponding to 5.85 pixels in this time. Hence the points of detection would be separated by 262+5.85±1 pixel rather than 262±1 pixel. In this case, the absence of a signal separated from the first point of detection by 262±1 pixel would signify that the passing label had in fact participated in an incorporation event.

To avoid smearing of the signal into adjacent pixels, it is preferred that the illumination provided to a label by the scanning laser persists for no longer than the time required for the label to move across 1 pixel; i.e., 50 microseconds. The thickness of the laser line required to give this duration of exposure is 0.5 μm+(5.0×10⁻⁵ s×1.95×10⁻³ m/s) =0.5 microns. Because the laser_scan speed is 250-pixels/0.064 s and the effective molecule flow speed is 250-pixels/0.0125 s, the laser scan would need to be repeated at intervals of 0.064+0.0125=0.0765 s to ensure that every passing label is detected. For a minimum camera exposure time of 0.350 s, the laser scan would be repeated at least 4.575 times for each frame. The exemplary values disclosed hereinabove may be achieved in conventional optical set up, and yield parameters that enable single molecule detection, by capturing the image of a single molecule onto an area the size of just one pixel. Methods and devices utilizing the exemplary values allow imaging of incorporation events for which the labeled moiety is stationary at the immobilized complex for at least about 1.5 milliseconds, preferably for at least about 0.1 millisecond, and preferably as long as about 1 second.

It will be recognized by those skilled in the art that the methods and devices disclosed herein can be used for identifying labels that are excited by a variety of wavelengths of light. For example, multiple lines of laser illumination at different colors may be used to scan the upstream illumination zone, so that each label is imaged at two separate locations in the first illumination zone, with the distance between the two detection points indicating the excitation color. Other embodiments will be apparent to one skilled in the art.

What is claimed is:

1. A method for detecting the incorporation of a nucleotide into a nucleic acid, comprising:
forming a solution comprising at least one NTP, wherein said NTP is labeled with a detectable label;
providing an immobilized complex comprising a target nucleic acid, a primer nucleic acid which complements a region of the target nucleic acid, and a nucleic acid polymerase enzyme molecule;
causing said solution comprising said labeled NTP to pass at a known speed through a first illumination zone upstream of the immobilized complex;
illuminating said labeled NTP with light at said first illumination zone, thereby causing said labeled NTP to emit light;
detecting said light emitted by said NTP at the first illumination zone and determining from said light the time at which said labeled NTP passes through the first illumination zone;
causing said solution comprising said labeled NTP to pass at a known speed past said immobilized complex, and through a second illumination zone downstream of said immobilized complex;
determining, from the known speed of said labeled NTP and from the time at which said labeled NTP passed through the first illumination zone a predicted time at which said labeled NTP would pass through the second illumination zone if said nucleotide were not incorporated into said immobilized complex;
detecting the amount of emitted light at said second illumination zone at said predicted time; and
determining from said amount of emitted light at said predicted time whether said nucleotide had become incorporated into the immobilized complex.

2. A method of claim 1 wherein the incorporation is detected without illuminating the complex.

3. A method of claim 1 wherein the incorporation is detected substantially without detecting said label while said label is in contact with the complex.

4. The method of claim 1 wherein said label is a fluorescent label.

5. The method of claim 1 wherein said label is attached to the beta or gamma phosphate of the NTP.

6. The method of claim 1 wherein the solution comprising said at least one NTP has a total NTP concentration of about 10⁻⁸ or less.

7. The method of claim 1 wherein said solution comprising said at least one NTP is caused to pass said illumination zones by electrokinetic flow.

8. A method of claim 1 wherein the nucelotide that is incorporated is identified.

9. A method of claim 8 wherein the nucleotide that is incorporated is identified by using a solution comprising only one type of labeled NTP.

10. A method of claim 1 wherein the nucleotide that is incorporated is identified by using a solution comprising differently labeled types of NTP and by determining the types of labeled NTPs detected in the first illumination zone by distinguishing the different optical signals produced by different labels.

11. A method of claim 10 wherein said different labels have different fluorescence emission wavelengths.

12. A method of claim 10 wherein said different labels have different fluorescence absorption wavelengths.

13. A method of claim 10 wherein said different labels have different fluorescence brightnesses.

14. A method of claim 8 wherein the detection and identification of incorporation of a nucleotide is repeated and the sequence of identified incorporations is collated to yield the sequence of a part of a nucleic acid, and thereby genotype or sequence the target nucleic acid.

15. A system for optically detecting incorporation of a nucleotide into an immobilized complex comprising a primer nucleic acid molecule and a polymerase enzyme molecule, comprising:
(a) a solution flow chamber having a surface bearing the complex;
(b) a solution for contacting the complex, said solution containing one or more labeled NTPs;
(c) an illuminating device for illuminating at least two regions nearby the complex with light, said illuminating device providing a first illumination zone and a second illumination zone in said solution flow chamber;
(d) a transporting device for transporting the labeled NTPs through said first illumination zone, then past said complex, and then through said second illumination zone;
(e) an optical detection device for collecting light signals from the first illumination zone, and for determining the times at which each labeled molecule transits through the first illumination zone;
(f) an optical detection device for collecting light signals from the second illumination zone; and
(g) a computational device, for determining when light signals would be expected from the second illumination zone from labeled molecules that were detected while transiting the first illumination zone.

16. A system for optically detecting incorporation of nucleotides into an array of immobilized complexes, each comprising a primer nucleic acid molecule and a polymerase enzyme molecule, comprising:
(a) a solution flow chamber having a surface bearing the array of complexes;
(b) a solution for contacting the array of complexes, said solution containing one or more labeled NTPs;
(c) an illuminating device for illuminating at least two regions nearby the array of complexes with light, said illuminating device providing a first illumination zone and a second illumination zone in said solution flow chamber;
(d) a transporting device for transporting the labeled NTPs through said first illumination zone, then past said array of complexes, and then through said second illumination zone;
(e) a means for translating at a known speed at least one of said illumination zones to provide a translating line of illumination at said solution flow chamber;
(f) an optical detection device for imaging light signals from the first illumination zone, and for determining the location and thereby the moment of time at which each labeled molecule transits through the first illumination zone;
(g) an optical detection device for imaging light signals from the second illumination zone; and
(h) a computational device.
